(19) United States
(12) Patent Application Publication
(10) Pub. No.: US 2025/0302881 A1
(43) Pub. Date: Oct. 2, 2025

(54) NANOMATERIAL-STEM CELL COMPOSITION AND METHODS OF USE

(71) Applicants: AION HEALTHSPAN, INC., Miami, FL (US); University of Miami, Miami, FL (US)

(72) Inventors: Giacomo Lanzoni, Miami, FL (US); Camillo Ricordi, Miami, FL (US); Dimitrios Kouropis, Miami, FL (US); Diana Velluto, Miami, FL (US)

(21) Appl. No.: 18/863,104

(22) PCT Filed: May 5, 2023
(86) PCT No.: PCT/US2023/021253

§ 371 Date: Nov. 5, 2024

Related U.S. Application Data
(60) Provisional application No. 63/338,593, filed on May 5, 2022.

(51) Int. Cl. A61K 35/28; A61K 9/70; A61K 31/439; A61K 35/39; A61K 49/00; A61P 3/10; C12N 5/0775

(52) U.S. Cl. CPC A61K 35/28; A61K 9/7007; A61K 31/439; A61K 35/39; A61K 49/0054; A61P 3/10; C12N 5/0668

(57) ABSTRACT

Disclosed herein are biocompatible and biodegradable nanomaterials combined with molecules of interest and stem cells in a variety of stable and safe compositions. The nanomaterials comprise poly(ethylene glycol)-oligo(ethylene sulfide) (PEG-OES) amphiphilic block-copolymers that self-assemble in supramolecular aggregates of fibrillar shape. The fibrillar architecture of the assemblies allows the easy, fast and not harmful internalization into stem cells, including the preferred umbilical cord derived mesenchymal stem cells (UC-MSC). The OES core enables loading of hydrophobic molecules, such as imaging agents and drugs, which are carried by the nFIB into the stem cells for a final product that comprises a composition of MSC, nFIB and therapeutic molecule (e.g., MSC-nFIB-Rapamycin). The technology can be utilized to enhance the immunoregulatory potency of MSC via intracellular nanomaterial delivery of immunosuppressive drugs, and to obtain active site-targeting and localized delivery of drug-loaded nanofibrils by exploiting the MSC homing ability.

### CROSS-REFERENCE TO RELATED APPLICATIONS

This application claims the benefit of U.S. Provisional Application No. 63/338,593, filed May 5, 2022, which is hereby incorporated by reference in its entirety.

### FIELD OF THE INVENTION

This disclosure relates generally to delivery of a molecule using nanomaterials and more specifically to the nanomaterial-drug delivery using mesenchymal stem cells.

### BACKGROUND

Nanotechnologies can provide innovative tools for local drug delivery by introducing biocompatible nanoparticle carriers. (Velluto and Ricordi, 2017; Duncan and Gaspar, 2011). Nanoparticles can load drugs and enable stable aqueous dispersions of poorly water-soluble therapeutic agents. Nanoparticles can also protect the drugs from degradation caused by endogenous mechanisms, which can reduce the dosage of the therapeutic agents and require increased frequency of administration. The nanoparticles can also provide sustained and localized drug release. Furthermore, the extremely small size and large surface area of nanoparticles can allow them to readily enter the cells in vitro and in vivo (Velluto et al., 2021) to enhance various molecular changes by delivering drugs, proteins, genes, or imaging agents.

However, systemic administration of drug-loaded nanoparticles implies the indiscriminate diffusion of the therapeutic agents throughout the body. This can result in reduced availability at the site of interest.

It would be advantageous to utilize cell therapy to target nanoparticle carriers to a particular location.

### SUMMARY OF THE INVENTION

This disclosure relates to a nanomaterial-stem cell composition comprising: a poly(ethylene glycol)-oligo(ethylene sulfide) PEG-OES based fibril nanomaterial (nFIB) incorporated into one or more stem cells. In an embodiment, the one or more stem cells are mesenchymal stem cells (MSC). In an embodiment, the MSC are derived from an umbilical cord. In an embodiment, the nFIB is comprised of a poly(ethylene glycol) (PEG) block molecular weight of 500-4,600. In an embodiment, the nFIB is comprised of an oligo(ethylene sulfide) (OES) with a degree of polymerization from 2 to 20. In an embodiment, the nFIB is comprised of a PEG block molecular weight of about 2000 and an OES block degree of polymerization of about 5. In an embodiment, the nFIB is PEG₄₄-OES₅. In an embodiment, the composition also comprises a molecule of interest. In an embodiment, a mass ratio of the molecule of interest to the nFIB is 10-30. In an embodiment, a mass ratio of molecule of interest to the nFIB is 20. In an embodiment, the solubility of the molecule of interest into the nFIB is about 0.1 mg/ml to about 20 mg/ml. In an embodiment, the solubility of the molecule of interest into the nFIB is about 3 mg/ml. In an embodiment, the molecule of interest is a drug or probe. In an embodiment, the probe is an imaging probe. In an embodiment, the molecule of interest is present in the mesenchymal stem cells. In an embodiment, the nFIB comprises a non-covalently attached molecule of interest. In an embodiment, the molecule of interest is covalently attached to the nFIB. In an embodiment, the molecule of interest is an anti-cancer drug. In an embodiment, the molecule of interest is radioactive. In an embodiment, the composition also comprises pancreatic islets or stem cell-derived islets. In an embodiment, the pancreatic islets are aggregated with the MSC-nFIB. In an embodiment, the nFIB is about 5 nm in diameter. In an embodiment, the nFIB is about 500 nm to 1.5 μm in length. In an embodiment, the nFIB is about 1.0 μm in length. In an embodiment, the nFIB minimally or does not alter the MSC phenotype or viability. In an embodiment, the molecule of interest is a hydrophobic therapeutic molecule. In an embodiment, the hydrophobic therapeutic molecule is rapamycin (RAPA). In an embodiment, the concentration of rapamycin between about 1.0 to 10.0 μg/mL. In an embodiment, the nFIB and rapamycin minimally or does not alter the MSC phenotype or viability. In an embodiment, the composition is utilized as a therapeutic, diagnostic, drug delivery mechanism, or extended release drug delivery mechanism. In an embodiment, the therapeutic is rapamycin (RAPA). In an embodiment, the diagnostic is a probe.

This disclosure relates to a method for preparing MSC-nFIB-molecule of interest comprising providing a PEG-OES copolymer; suspending the PEG-OES copolymer and a molecule of interest in water or an organic solvent; removing unloaded molecule of interest; adding the PEG-OES-molecule of interest to MSC; and incubating the PEG-OES-molecule of interest and the MSC together. In an embodiment, the molecule of interest is rapamycin (RAPA). In an embodiment, the molecule of interest is a probe. In an embodiment, the probe is fluorescent. In an embodiment, the MSC are derived from an umbilical cord. In an embodiment, the incubating occurs for 1, 2, 4, 6, 12, 18, 24, 36, 40, or 48 hours. In an embodiment, the incubating occurs for 24 hours.

This disclosure relates to a method of treating a condition in a subject comprising administering MSC-nFIB-molecule of interest to the subject in need thereof. In an embodiment, pancreatic islet cells are also administered. In an embodiment, the condition is diabetes. In an embodiment, the molecule of interest is rapamycin (RAPA) and MSC-nFIB-RAPA is formed. In an embodiment, the MSC-nFIB-molecule of interest is injected into the subject as a therapeutic concentration. In an embodiment, the MSC-nFIB-RAPA reduces the proliferation of cytotoxic T cells when the cytotoxic T cells are in proximity to the MSC-nFIB-RAPA or derivatives. In an embodiment, the MSC-nFIB-RAPA expands regulatory T cells when the regulatory T cells are in proximity to the MSC-nFIB-RAPA or derivatives. In an embodiment, the MSC-nFIB-molecule of interest reaches a site of inflammation after intravenous infusion. In an embodiment, the MSC-nFIB-molecule of interest are localized at a site of inflammation or implantation. In an embodiment, the MSC-nFIB-molecule of interest release nFIB and/or a drug over time. In an embodiment, the MSC-nFIB-molecule of interest are aggregated with pancreatic islet cells or other cells. In an embodiment, the MSC-nFIB-molecule of interest are co-transplanted in a confined space and remain in or in proximity to such confined space for at least 7 days. In an embodiment, the MSC-nFIB-molecule of interest are aggregated and integrated with or on a surface of pancreatic islets and the functionality of the islets in vivo is not affected. In an embodiment, the MSC-nFIB-molecule of interest are intended for therapeutic use. In an embodiment, the MSC-nFIB-molecule of interest, further provide a diagnostic ability. In an embodiment, the MSC-nFIB-molecule of interest provide drug delivery. In an embodiment, the MSC-nFIB-molecule of interest provide extended release of a drug. In an embodiment, the MSC-nFIB-molecule of interest modulate immune functions. In an embodiment, the MSC-nFIB-molecule of interest improve the outcomes of a transplant.

This disclosure relates to a kit comprising: instructions for using a nanomaterial-stem cell composition comprising an nFIB incorporated into MSC; and the nanomaterial-stem cell composition comprising an nFIB incorporated into MSC.

This disclosure relates to a kit comprising: instructions for performing the method of any one of claims 32-42; and a nanomaterial-stem cell composition comprising an nFIB incorporated into MSC.

The present technology provides for a nanomaterial-stem cell composition that combines nanomaterials, cell therapy, and drugs, based on poly(ethylene glycol)-oligo(ethylene sulfide) (PEG-OES) nanofibril drug delivery systems and Mesenchymal Stem Cells (MSC). The present technology addresses internalization of drug-loaded nanofibrils into the MSC and co-administration. The ultra-small diameter of the nanofibrils can enable their incorporation in the MSC, which can be especially efficient because the extra-long nanofibril shape can allow multi-anchorage points of nanofibrils on the cell surface, resulting in facilitated cellular uptake. The hydrophobic core, which can be made by the OES block, also can enable stable loading of hydrophobic molecules in the nanofibrils and their storage into the MSC. In one embodiment of the technology, the hydrophobic immunosuppressive drug Rapamycin can be loaded in the PEG-OES nanofibril drug delivery system and combined with the cells.

The present technology further relates to nanomaterial-combined cell therapy and its uses to improve the efficacy and increase the safety of anti-inflammatory and anti-rejection treatments. The present technology can enhance the immunoregulatory potency of MSC via intracellular nanoparticle delivery of immunosuppressive drugs (ISDs) and it can exploit the MSC homing ability to obtain active site-targeting of drug-loaded nanoparticles. In other embodiments, the technology could also be applied to anticancer uses, in which an appropriate anticancer drug could be delivered to a tumor site exploiting the MSC homing ability and obtaining active site-targeting of drug-loaded nanoparticles.

The nanoparticles can be prepared to internalize a hydrophobic drug, such as Rapamycin, or imaging reagents, such as lipophilic molecules.

In a first aspect, the technology can provide the method and the block copolymer for efficient and durable internalization of nanoparticles into stem cells in a short time and with minimal or negligible effects on cell viability and phenotype. The block copolymers for this invention can be selected to form nanofibrillar architecture and nanofibrillar networks by supramolecular self-assembling in water and, for the latter, by entanglement of the nanofibrils. Some embodiments can contain linear copolymers made of the hydrophilic block PEG with molecular weight 2,000. Other embodiments can contain PEG of molecular weight 4,600. In an embodiment, the molecular weight of PEG is about 100, about 200, about 300, about 400, about 600, about 800, about 1000, about 1200, about 1400, about 1600, about 1800, about 2000, about 2200, about 2400, about 2600, about 2800, about 3000, about 3200, about 3400, about 3600, about 3800, about 4000, about 4200, about 4400, about 4600, about 4800, about 5000, about 5200, about 5400, about 5600, about 5800, or about 6000. Yet other embodiments can contain the hydrophobic, highly crystalline, block OES with a degree of polymerization from about 2 to 20. In an embodiment, the degree of polymerization is from about 5 to 10. In an embodiment, the degree of polymerization is 2, 3, 4, 5, 6, 7, 8, 9, 10, 11, 12, 13, 14, 15, 16, 17, 18, 19, or 20. In an embodiment, the degree of polymerization is 5. In some block copolymers, a moiety selected from a fluorophore can be conjugated to the hydrophobic block OES.

The method can include combination of nanoparticles with cells. In an embodiment, the cells are stem cells. In an embodiment, the stem cells are at least one of hematopoietic stem cells, mesenchymal stem cells, neural stem cells, epithelial stem cells, and skin stem cells. In another embodiment, the cells are MSCs. In an embodiment, the cells are derivatives of stem cells. In an embodiment, MSCs can be derived from human umbilical cord. In other embodiments, MSC can be derived from tissues such as adipose tissue, bone marrow, amniotic membrane, or placenta. In a preferred embodiment MSCs can be prepared in adhesion to tissue culture-treated plastic and cultured in an aqueous solution. In other embodiments MSCs can be prepared in adhesion to another matrix or resuspended in an aqueous solution.

Nanoparticles can be dispersed in a liquid carrier and ultimately in the same aqueous solution of cells. The aqueous solution can enable the contact between the nanoparticles and the MSCs. Therefore, the embodiments can contain stem cells fortified with PEG-OES based nanofibrils (MSC-nFIB), loaded with one or more drugs and/or imaging agents.

In a second aspect, the technology can provide the method for delivering a therapeutic concentration of pharmaceutically relevant molecules and biomolecules into MSCs and improves their therapeutic features. The method can include providing the block copolymer nanofibrils (of the first aspect) containing a therapeutic molecule and contacting the MSCs (MSC-nFIB-D, where D is a therapeutic molecule), thereby being internalized by the cells and delivering the molecule to the cells. In some embodiments, the therapeutic molecule can be covalently attached to the nanofibrils. In other embodiments, the therapeutic molecule can be a hydrophobic molecule dissolved or dispersed in the nanofibrils. Furthermore, the nanomaterials can be a depot deposited or administered with the MSC (MSC/nFIB-D). In this invention the nanomaterial-MSC compositions can be for use as medicaments. This can result in enhanced immunomodulatory properties compared to mesenchymal stem cells alone or nanomaterial alone.

In a third aspect, the technology can provide the method for active site-targeting of drug-loaded nanoparticles. The method can include the use of MSCs as carriers for the nanofibrils and for homing them to the site of interest. In some embodiments the MSCs can incorporate nanofibrils of the second aspect (loaded with therapeutic molecules) and can be used to carry/transport them to the site of inflammation/injury to deliver the therapeutic agents. In some embodiments the MSCs incorporate nanofibrils of the second aspect and can be used as a localized deposit of nanoparticles at the site of interest. In this aspect, the nanomaterial-MSC compositions can be used as a medicaments for targeted and localized drug delivery to lower or minimize side effects.

In a fourth aspect, the technology can provide nanomaterial-MSC, and drug compositions combined with pancreatic islet cells as a method of protecting cells from host rejection for the enhancement of cell replacement therapies. In some embodiments the nanomaterial-MSC composition can contain a fluorescent probe for MSC and for the nFIB core for traceability. In other embodiments they can contain therapeutic molecules (in one embodiment: Rapamycin) loaded into the nanomaterial nFIB (as of the third aspect). In this aspect, the technology nanomaterial-MSC compositions can be for use as a medicaments in cell transplantations.

In a fifth aspect, the technology can provide a pharmaceutical formulation of the nanomaterial-stem cell compositions, in combination with a drug, in a pharmaceutically acceptable liquid carrier, which can be maintained viable in culture or that can be cryopreserved and subsequently thawed for use on demand.

### BRIEF DESCRIPTION OF THE DRAWINGS

FIG. 1 depicts a schematic of the invention showing nanomaterial-stem cells composition for drug delivery uses.

FIG. 2A depicts a confocal microscope image of the nanomaterial-stem cells composition.

FIG. 2B depicts a quantification of fluorescent signal intensity of the nanomaterial-stem cell composition via flow cytometry analysis of stem cells.

FIG. 3A depicts nanomaterial-stem cells composition as shown by the presence of bright fluorescent dots of core-labeled nanofibrils in the cytoplasm after 24 and 48 hours of nanomaterial-cell contact.

FIG. 3B depicts the CCK-8 assay of unloaded (black bars) or loaded with the molecule drug Rapamycin (RAPA, silver bars) at 24 and 48 hours.

FIG. 3C depicts nanomaterial-stem cells composition as shown by the presence of fluorescent dots of core-labeled nanofibrils in the cytoplasm after 120 hours of nanomaterial-cell contact.

FIG. 4A depicts immunophenotypic profile of naïve and TNFα, IFNγ, CTGF (TIC)-treated stem cells and nanomaterial-stem cells with various antibodies.

FIG. 4B depicts immunophenotypic profile of naïve and TNFα, IFNγ, CTGF (TIC)-treated stem cells and nanomaterial-stem cells with various antibodies.

FIG. 5A depicts flow cytometry zebra-plots that show the CD4+ T cell proliferation via CellTrace dilution after contact co-culture with nanomaterial-stem cell compositions and control stem cells.

FIG. 5B depicts CD4 T cells proliferation index calculated for each nanomaterial-stem cell composition.

FIG. 5C depicts flow cytometry zebra-plots that show the CD8+ T cell proliferation via CellTrace dilution for each nanomaterial-stem cell composition.

FIG. 5D depicts proliferation indexes calculated for each nanomaterial-stem cell composition.

FIG. 6A depicts regulatory T cells (Treg) staining analyzed via flow cytometry after coculture of T cells with Mesenchymal Stem Cells alone (stem cells are UC-MSC).

FIG. 6B depicts regulatory T cells (Treg) staining analyzed via flow cytometry after coculture of T cells with drug loaded nanomaterial-Mesenchymal Stem Cell composition (MSC-nFIB-RAPA).

FIG. 6C depicts mean fluorescent Intensity (MFI) of the Treg marker FoxP3 in the gated CD4+, CD25+, and FoxP3+ cell population and reported as percentage increase comparted to (control is Treg in co-culture with stem cells alone) for nanomaterial-stem cell compositions.

FIG. 7 depicts a confocal microscopy image shows the stability of nanomaterial stem cells composition after the preparation for applications in vivo.

FIG. 8A depicts results of an In Vivo Imaging System (IVIS) to detect the accumulation of MSC-nFIB composition, administered by intravenous infusion, in vivo in BALB/c mice that were treated with Lipopolysaccharide (LPS) injection in the right foot paw to induce localized inflammation.

FIG. 8B depicts results of an In Vivo Imaging System (IVIS) to detect the accumulation of MSC-nFIB composition, administered by subcutaneous infusion, in vivo in BALB/c mice that were treated with Lipopolysaccharide (LPS) injection in the right foot paw to induce localized inflammation.

FIG. 8C depicts results of an In Vivo Imaging System (IVIS) to detect the accumulation of MSC-nFIB composition, administered by subcutaneous infusion, in vivo in BALB/c mice that were treated with Lipopolysaccharide (LPS) injection in the right foot paw to induce localized inflammation after a period of fifty days.

FIG. 9A depicts a schematic of the MSC-nFIB composition and its use as drug delivery system in pancreatic islet transplantation.

FIG. 9B depicts a fluorescence microscopy image shows MSC-nFIB composition aggregated with mouse islets after overnight co-culture.

FIG. 9C depicts a drawing representation of the epidydimal fat pad (EFP) as a site of choice for islet transplant in mice.

FIG. 10A depicts in vivo imaging of mice that received implantation of syngeneic pancreatic islets, untreated or pre-aggregated with MSC or with MSC-nFIB compositions, in the epidydimal fat pad (EFP) at post operative day 7.

FIG. 10B depicts in vivo imaging of mice that received implantation of syngeneic pancreatic islets pre-aggregated with MSC or with MSC-nFIB compositions, in the epidydimal fat pad (EFP), at post operative day 7.

FIG. 10C depicts ex vivo imaging of the EFP resected from mice implanted with syngeneic pancreatic islets either alone, pre-aggregated with MSC, or pre-aggregated with MSC-nFIB compositions where the MSC were stained with a fluorescent probe (DiD).

FIG. 10D depicts ex vivo imaging of the EFP resected from mice implanted with syngeneic pancreatic islets alone or pre-aggregated with MSC, or pre-aggregated with MSC-nFIB compositions where the nFIB contain a fluorescent core-labeling dye (DiR).

FIG. 11 depicts blood glucose levels (BGL) of a set of C57BL/6 mice that were rendered diabetic via streptozotocin treatment with pancreatic islets were isolated from healthy donor C57BL/6 mice for syngeneic transplantation in diabetic C57BL/6 mice.

### DETAILED DESCRIPTION

The foregoing aspects, features, and advantages of the present technology will be further appreciated when considered with reference to the following description of preferred embodiments and accompanying drawings, wherein like reference numerals represent like elements. In describing the preferred embodiments of the technology illustrated in the appended drawings, specific terminology will be used for the sake of clarity. The present technology, however, is not intended to be limited to the specific terms used, and it is to be understood that each specific term includes equivalents that operate in a similar manner to accomplish a similar purpose.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment,” “an embodiment,” “certain embodiments,” or “other embodiments” of the present invention are not intended to be interpreted as excluding the existence of additional embodiments that also incorporate the recited features. Furthermore, reference to terms such as “above,” “below,” “upper,” “lower,” “side,” “front,” “back,” or other terms regarding orientation are made with reference to the illustrated embodiments and are not intended to be limiting or exclude other orientations.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “subject” as used herein refers to any animal, such as any mammal, including but not limited to, humans, non-human primates, rodents, mammals commonly kept as pets (e.g., dogs and cats, among others), livestock (e.g., cattle, sheep, goats, pigs, horses, and camels, among others) and the like. In some embodiments, the mammal is a mouse. In some embodiments, the mammal is a human.

The term “injection” as used herein includes, but is not limited to, intravenous (IV) injections, intramuscular (IM) injections, subcutaneous (SC) injections, and intradermal (ID) injections.

Drug-loaded nanoparticles can be combined with cells to improve therapeutic outcomes. Mesenchymal Stem Cells, also known as Mesenchymal Stromal Cells or Medicinal Signaling Cells (MSCs) (Uccelli A. et al., 2008) possess powerful features for therapeutic purposes, mainly thanks to their capability to migrate to the site of inflammation (chemotaxis) (Anthony et al., 2013; Fox et al., 2007), accelerate tissue regeneration, and support tissue homeostasis by enhancing beneficial functions of endogenous cells. MSCs also have immunomodulatory and anti-inflammatory functions (Yagi et al., 2010). In vitro, MSCs inhibit T cell proliferation and promote regulatory T cell expansion and function (Le Blanc et al., 2007; De Miguel et al., 2012). In vivo, MSCs show a very good safety profile when administered intravenously (Thompson et al., 2020) and have been shown to modulate alloimmune response in models of islet and heart transplantation (Eggenhofer et al., 2011; Ding et al., 2009; Berman et al., 2010). Although the properties of MSCs make them attractive for treating inflammatory conditions, they can act in a transient manner (Girdlestone J. et al., 2015), which can make them less convenient for many medical conditions (e.g., anti-rejection therapies).

Therefore, nanotechnologies can be designed and developed to improve the therapeutic outcome of stem cell-based therapies. This can be accomplished by using biodegradable nanoparticle-based systems for the provision of bio-chemical, physical and genetic cues to control MSC behavior and enhance their beneficial properties. This can further use nanoparticle enhanced structured scaffolds and surfaces to recapitulate the stem cell niche within a tissue. Additionally, this can include nanoparticle encapsulation into MSC as carriers to monitor their effectiveness in vivo and for the specific delivery of therapeutic agents to a desired site (e.g., site of inflammation, tumor, or graft implantation site).

Targeted delivery of drugs is a promising field in anticancer therapy. MSCs can have inherent tumor-tropic and migratory properties, which can allow them to serve as vehicles for targeted drug delivery systems for isolated tumors and metastatic diseases. MSCs have been successfully studied and discussed as a vehicle for cancer gene therapy but not for delivery of traditional anticancer drugs.

Some studies (Sadhukha et al., 2014) have shown that poly(lactic-co-glycolic acid) PLGA nanoparticles can be internalized into cells through endocytosis and a fraction of the internalized particles escape the endo-lysosomal pathway to reach the cytoplasm. Nanoparticles that are retained within the cells can act as intracellular drug depots, slowly releasing the encapsulated drug. Thus, MSCs loaded with drug-containing nanoparticles (nano-engineered MSCs) can be capable of actively accumulating in tumors and slowly releasing the drug, resulting in effective inhibition of tumor growth.

Considering the limiting number of MSCs targeting the desired site (e.g., microenvironment of the tumor, or inflamed site, or other site), it can be critical to have enough drug-loaded nanoparticles incorporated into the MSCs and to achieve a therapeutic drug concentration in the target tissue(s). The internalization of nanoparticles (NPs) can be improved by modification of the NPs, size control, proper incubation time, and NP concentration. NPs can be modified by conjugation of antibodies or addition of cationic moieties on their surface to improve cellular internalization. Such alterations need to be carefully evaluated, because they could cause higher toxicity.

Nanoparticles can be prepared by different methods. Among those available, supramolecular self-assembly of amphipathic block copolymers can be utilized to generate nano- and microscale materials with controllable architectures. By controlling block and polymer characteristics including chemical composition, relative hydrophobicity and hydrophilicity, and absolute and relative block size, it can be possible to obtain different architectures such as spherical micellar, linear fibrillar, and spherical vesicular architectures. The chemical composition of the molecules can be critical to enable self-assembly for the relative application. For biological applications, most of the polymer-based molecules can contain the biocompatible polymer poly(ethylene glycol) (PEG). The presence of PEG in the surface of nanoparticles enhances their lifetime in the biological environment and limits their recognition by phagocytes upon injection or implantation.

For the preparation of block copolymer amphiphiles that yield micellar, fibrillar, and vesicular architectures upon self-assembly (Cerritelli et al., 2009; Velluto et al., 2008), PEG-based macroinitiators can be utilized for ring-opening polymerizations of the monomer propylene sulfide. For the preparation of block copolymer amphiphiles that uniquely yield a fibrillar architecture with ˜ 5 nm diameter and >500 nm length upon self-assembly, PEG-based macroinitiators can be utilized for ring-opening oligomerizations of the monomer ethylene sulfide (Brubaker et al., 2015).

For drug-delivery purpose, nanoscopic fibrils (nanofibrils, nFIB) made by self-assembling of the block-copolymer poly(ethylene glycol)-oligo(ethylene sulfide) (PEG-OES) in a hot water suspension, are particularly useful. The small diameter (5 nm) enables the nanofibrils to be internalized into a variety of cells, including immune cells in vitro and in vivo, and their elongated shape (about 1 μm length), not only can enhance cellular uptake but also can provide better localized drug delivery than spherical nanoparticles. This is because the nanofibrils can reduce their passive transport under fluid flow conditions promoting local retention. Like micellar nanoparticles, the nanofibrils can load hydrophobic drugs enabling their stable aqueous dispersions, therefore reducing their dosage and their side effects. They can also provide sustained drug release.

Biocompatible and biodegradable nanomaterials combined with therapeutic molecules and stem cells in a variety of stable and safe compositions can be used as medicaments. The technology comprises poly(ethylene glycol)-oligo(ethylene sulfide) (PEG-OES) amphiphilic block-copolymers that self-assemble in supramolecular aggregates of fibrillar shape. The fibrillar architecture of the assemblies, named nanofibrils (nFIB) because of their extremely small diameter (5 nm), allows the easy, fast and not harmful internalization into stem cells, including the preferred umbilical cord derived mesenchymal stem cells (UC-MSC). Furthermore, the OES core enables loading of hydrophobic molecules, such as imaging agents and drugs, which are carried by the nFIB into the stem cells for a final product the comprises a composition of MSC, nFIB and a molecule of interest, such as a therapeutic molecule (e.g., MSC-nFIB-Rapamycin). The technology is for use to enhance the immunoregulatory potency of MSC via intracellular nanomaterial delivery of immunosuppressive drugs, and to obtain active site-targeting and localized delivery of drug-loaded nanofibrils, by exploiting the MSC homing ability. In an embodiment, the molecule of interest is one or more of a drug, protein, gene, probe, radioactive agent, label, and imaging agent.

The present technology describes a nanomaterial-stem cell composition, in combination with a drug of interest, to modulate overactive immune and hyper-inflammatory processes happening at the site of a transplant, without affecting any other organ or tissue, and without systemic immunosuppression. In particular, mesenchymal stem cells can be fortified with PEG-OES based nanofibrils (MSC-nFIB), loaded with one or more drugs and/or imaging agents. The present technology demonstrates that the nanofibrils, either loaded or unloaded with small molecules, can be efficiently internalized in the cells and remain stable for days without affecting the cells phenotype and viability. The present technology demonstrates that the homing ability of the MSC allows targeted release of drug-nanofibrils by using the MSC as carriers, and/or localized release of drug-nanofibrils by using the MSC as a depot deposited at the site of interest. The present technology demonstrates that the drug-nanofibrils or the drugs are released from the MSC over several days in vivo at the target site.

Common anti-cancer drugs include, but are not limited to, cisplatin, erdafitinib, fluorouracil, mitomycin, gemcitabine, methotrexate, vinblastine, doxorubicin, paclitaxel, rapamycin, and combinations thereof. Particular combinations include, but are not limited to, cisplatin+fluorouracil, fluorouracil+mitomycin, cisplatin+gemcitabine, cisplatin+methotrexate+vinblastine, cisplatin+methotrexate+vinblastine+doxorubicin, and gemcitabine+paclitaxel.

The fibril architecture can be a convenient morphology to obtain high internalization of nanomaterials into the cells. In the embodiments, the fibrils can be made of an amphiphilic block-copolymer poly(ethylene glycol)-oligo(ethylene sulfide) (PEG-OES), where the PEG molecular weight is 2,000 and the number of OES units are 5 (PEG₄₄OES₅). The self-assembling of PEG-OES can be obtained in a liquid carrier by simple resuspension at warm temperature or by cosolvent evaporation method in the presence of an organic solvent. In an embodiment, the minimum amount of copolymer is from about 40 mg/mL up to about 160 mg/mL that serve as stock solutions ready to use, as they are stable for months. In an embodiment, the amount of copolymer is about 1 mg/ml, about 5 mg/ml, about 10 mg/ml, about 20 mg/ml, about 40 mg/ml, about 60 mg/ml, about 80 mg/ml, about 100 mg/ml, about 120 mg/ml, about 1140 mg/ml, about 160 mg/ml, about 200 mg/ml, or about 300 mg/ml. To enable the contact between the nanofibrils the MSC, and their cell internalization, the nanofibrils can be directly diluted to the desired concentration in the same aqueous solution where the MSC are cultured.

In certain embodiments, the nanofibrils can be formed by a hot water resuspension method without the need of organic co-solvents, which can make the formulation easier to prepare and safer for uses in biological environments. Because of the hydrophobicity of the OES core, the nanofibrils can efficiently incorporate small hydrophobic molecules by mixing those molecules with the block copolymer before resuspension in water. Alternatively, one or more hydrophobic small molecules can be dissolved in the organic solvent phase used to prepare nanofibril assemblies through emulsion solvent evaporation or thin film evaporation. In an embodiment, one or more small hydrophobic molecules can be represented by an imaging agent or a pharmaceutically-relevant small molecule drug; they do not interfere with or prevent assembly. Small molecules can get internalized into the MSC within the nanofibrils and don't lose their functional properties, but their solubility, safety, and efficacy are improved. In an embodiment, the drug molecule can be the hydrophobic immunosuppressant Rapamycin, with solubility into the nanofibrils up to 3 mg/mL depending on the copolymer/drug ratio used. The polymer/drug (mg/mg) ratio used in an embodiment is 20 mg/mg.

Different ratios between nanomaterials, cells and small molecule drugs can be used to prepare a variety of compositions. In various embodiments, the composition contains a ratio between the molecule of interest and the MSC or MSC-nFIB of about 1, about 3, about 5, about 7, about 10, about 13, about 15, about 17, about 20, about 23, about 25, about 27, about 30, about 35, about 40, about 50, about 60, about 70, about 80, about 90, or about 100. Various embodiments contain nFIB-RAPA dispersed in the culture solution of the MSC at a concentration of Rapamycin between about 1.0 to 10.0 μg/mL (1.0 to 10.0 mg/L) when the MSC are at 65% confluency. The product can be harvested after 24 hours. The MSC-nFIB-RAPA compositions can be stable in solution and can be cryopreserved and be ready to use on demand. Furthermore, the compositions reduce human cytotoxic T cell proliferation in vitro, and expand regulatory T cell in a more efficient way than MSC alone or nanofibrils alone.

Nanomaterial stem cells composition containing imaging agents can also be included in the disclosure and can be used for in vivo imaging purpose. Both the nanomaterials and the cells can contain a fluorescent probe with separated emission wavelengths that can distinguish the nFIB versus the MSC when they are combined. An embodiment can be made with MSC bearing a cellular probe DiD (also named DiIC18(5); 1,1′-dioctadecyl-3,3,3′,3′-tetramethylindodicarbocyanine perchlorate) and nFIB containing a core labeling dye DiR (also named DiIC18(7) (1,1′-dioctadecyl-3,3,3′,3′-tetramethylindotricarbocyanine iodide)). Fluorescent MSC-nFIB composition can accumulate in the inflammation site in mice that were previously treated with an injection of Lipopolysaccharide (LPS) in the right foot paw. The injection can be administered via intravenous or subcutaneous infusion. A variety of cellular probes and fluorescent dyes can be included in the MSC and nFIB respectively as needed. The nFIB can be released at the site of inflammation for up to 50 days. Because of their small diameter, the nFIB, once released from the MSC, can accumulate in the immune cells of the draining lymph nodes where their payloads of choice, once delivered, can regulate the local immune response.

In a cell transplantation model, a set of C57/BL6 mice can be turned diabetic by administration of streptozotocin and can receive implantation of syngeneic pancreatic islets on the epidydimal fat pad (EFP). Fluorescently labeled MSC-nFIB, can be previously aggregated on the surface of the islets and the final composition Islets-MSC-nFIB implanted into the selected site (EFP). In various embodiments, the composition contains a ratio between the islet cells and the MSC or MSC-nFIB of about 1 to 100. In various embodiments, the composition contains a ratio between the islet cells and the MSC or MSC-nFIB of about 5 to 20. In various embodiments, the composition contains a ratio between the islet cells and the MSC or MSC-nFIB of about 1, about 3, about 5, about 7, about 10, about 13, about 15, about 17, about 20, about 23, about 25, about 27, about 30, about 35, about 40, about 50, about 60, about 70, about 80, about 90, or about 100. MSC-nFIB composition do not negatively affect the anti-diabetic beta cell function of islet grafts because diabetes can be reversed in all recipient mice for the duration of the follow-up. The instant invention demonstrated that the MSC-nFIB can be retained at the site of transplant, which in various embodiments is the EFP, for at least 7 days. Therefore, the instant technology can be used as a powerful tool for localized and sustained drug delivery systems in pancreatic islet transplantation.

Various embodiments can be administered via IV infusions, other embodiments can be administered via SC injection close to the site of interest, and yet other embodiments can be transplanted together with the cells, either in the EFP or any other area of the body suitable for receiving an implantation. Other body areas can include kidney capsule, liver, subcutaneous space, intramuscular space.

The various embodiments can be cryopreserved and ready to use after thawing. Therefore, in an embodiment, the pharmaceutical formulation for clinical uses can consist only of any aqueous solution suitable to resuspend the MSC-nFIB composition. In an embodiment, other substances can be present in the pharmaceutical formulation.

### First and Second Medical Uses

The therapy disclosed herein involves one or more compounds. The disclosure relates to use of any compound described herein in therapy, including, but not limited to, therapy for diabetes, cancer, organ transplant, as an anti-inflammatory, or any condition in which the subject would benefit from the immune system being suppressed or regulated. The disclosure also relates to any use of any compound described herein for the manufacture of a medicament.

### Kits

Instructions for performing any method disclosed herein may be packaged with one or more of the reagents or components used in that method. Such a packaging of the instructions and the reagents or components may be termed a “kit.”

The instructions may be packaged in the kit in the form of printed instructions, a printed document providing a uniform resource locator (URL) from which detailed instructions may be accessed upon a user's entry of the URL into the address bar of a web browser, a printed document providing a Quick Response (QR) code which can be scanned to direct a smartphone or tablet computer's browser to a URL, or the like.

Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as Styrofoam, etc., may be enclosed. An identifier, e.g., a bar code, radio frequency identification (ID) tag, etc., may be present in or on the kit or in or one or more of the vessels or containers included in the kit. An identifier can be used, e.g., to uniquely identify the kit for purposes of quality control, inventory control, tracking, movement between workstations, etc.

### EXAMPLES

### Example 1: Assessment of Nanomaterial Internalization into Stem Cells

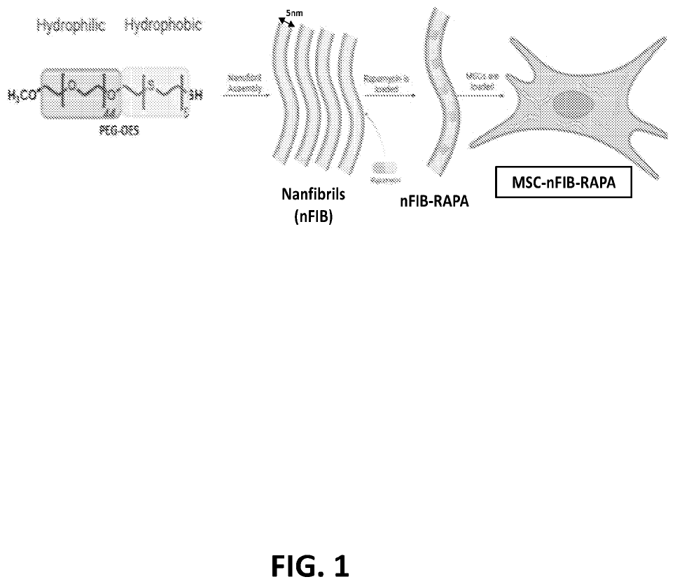

PEG-OES block copolymers, for which the chemical structure is schematized in FIG. 1, were synthetized and characterized. The fibril supramolecular assemblies (nFIB, FIG. 1) were obtained via resuspension of the copolymer in water using the method of the hot water emulsion. In particular, FIG. 1 shows nanofibrils of PEG₄₄-OES₅ loaded with Rapamycin (RAPA) and incorporated into the MSCs by means of contact. In this example, the nanofibrils were prepared with 80 mg of PEG₄₄-OES₅ in 1 mL of water and stored at 4° C. The same method, but in the presence of a desired amount of Rapamycin, was used to prepare PEG-OES nFIB-RAPA, followed by removal of unloaded drug molecules via centrifugation/precipitation. For the preparation of fluorescent PEG₄₄-OES₅ nFIB, the lipophilic, near-infrared fluorescent cyanine dye DiR was resuspended in DCM (dichloromethane) and loaded into nFIB (40 mg/mL) by the cosolvent evaporation method at a final stock concentration of 5 to 10 μM. The nFIB-DiR were exhaustively dialyzed against deionized water to remove possible unloaded dye molecules.

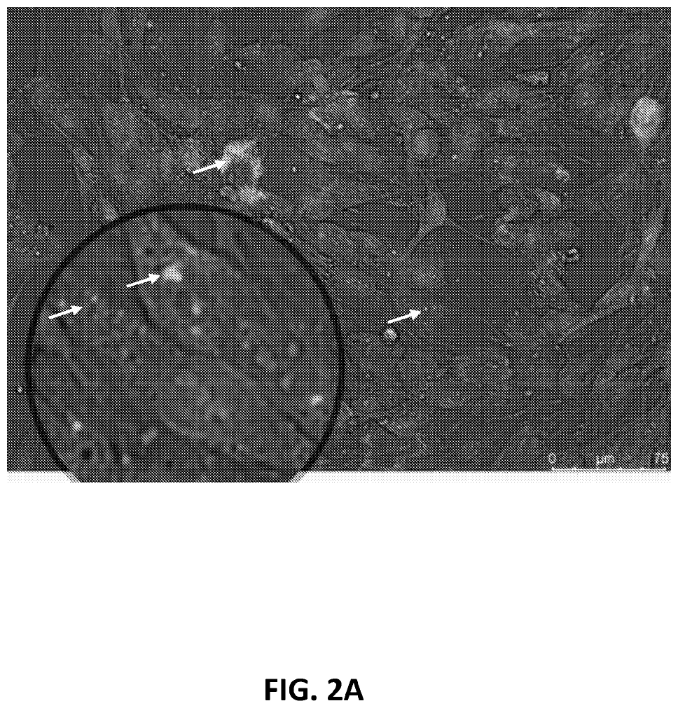

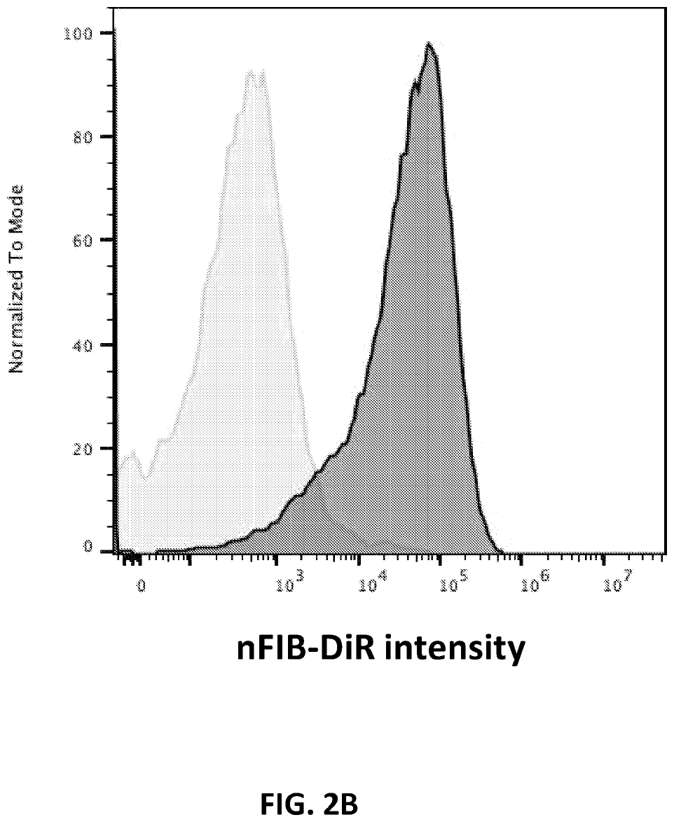

In this example, umbilical cord derived mesenchymal stem cells (UC-MSC) were culture-expanded from a previously established and characterized Master Cell Bank (MCB) derived from the subepithelial lining of a UC collected from a healthy term delivery. The samples containing nFIB-DiR, were used at dilution of 10⁴-folds for in vitro assessment of their internalization into the MSC. In this example, MSC were seeded at 40,000 cells/cm² and added with the labeled nFIB when they reached 65% confluency. The mixture was incubated for 24 hours, then the culture media was removed, cells were washed several times with PBS and fresh media was added. In FIG. 2A, the stem cells are umbilical cord derived mesenchymal stem cells (UC-MSC) and the nanomaterials are the nanofibrils of PEG₄₄-OES₅ with a fluorescent core-labeling lipophilic dye (DiR, also known as DiIC18(7); 1,1′-dioctadecyl-3,3,3′,3′-tetramethylindotricarbocyanine iodide)). Fluorescent microscopy showed the nFIB-DiR homogeneously internalized into the MSC (brighter dots in FIG. 2A, white arrows). Furthermore, nFIB internalization was evaluated using flow cytometry analysis (FIG. 2B) of the MSC untreated and treated with nFIB-DiR harvested and stained for LIVE/DEAD probe. The dark gray histogram of FIG. 2B is a fluorescent signal intensity of nanomaterial-stem cell composition prepared using nanomaterials with the fluorescent core-labeling dye DiR. The light gray histogram is the stem cells alone. Results showed that almost 100% of live MSC were positive for the DiR fluorescent signal, which means they have internalized the nFIB and they have formed a viable and stable MSC-nFIB composition.

### Example 2: MSC-nFIB and MSC-nFIB-RAPA Viability

Exploiting the homing ability of the MSC, the current technology allows targeted release of drug-nanofibrils by using the MSC as carriers to transport drug-loaded nFIB to the site of injury/transplant and release the drug over a period of several days.

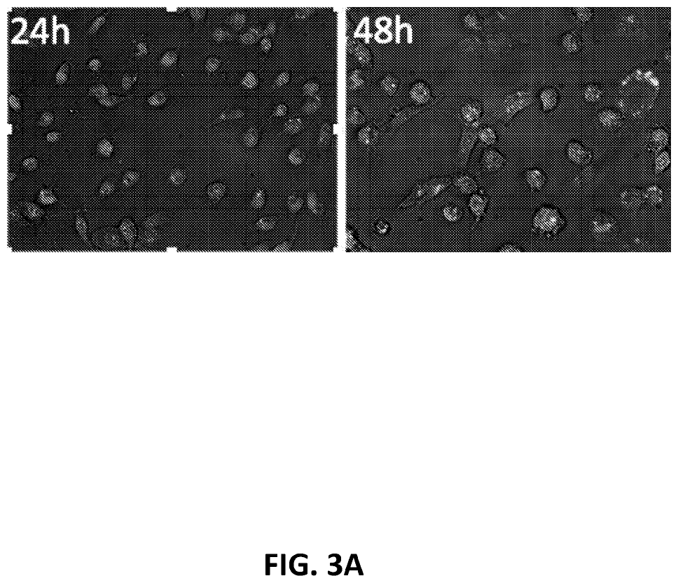

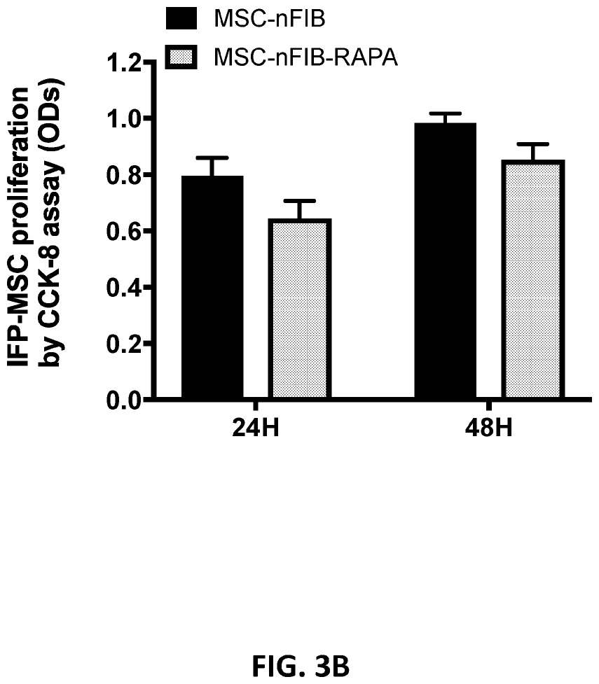

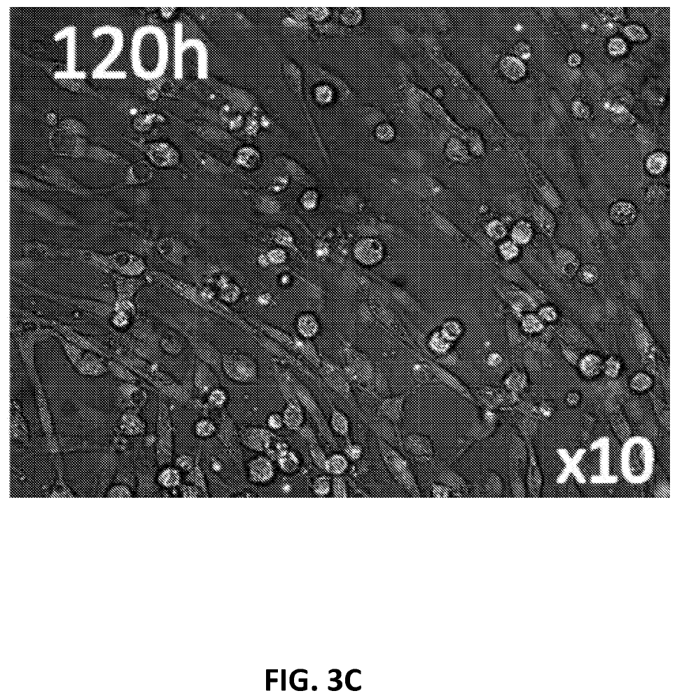

In this example, human infrapatellar fat pad-derived mesenchymal stem cells (IFP-MSC) were used. Cells were seeded at 10⁴ cells/well in a 24-well plate in contact with fluorescent PEG₄₄-OES₅ nFIB, prepared as in Example 1 and diluted 103-folds in the MSC solution. Fluorescent microscopy images show internalization of nFIB into the MSC 24 hours later, which continues also after 48 hours (FIG. 3A) without interfering with cell proliferation. The images of FIG. 3A were obtained by optical fluorescent microscopy in the channel for a fluorescent core-labeling lipophilic dye (DiR) present in the nanofibrils. CCK-8 cytotoxicity assay (Cell Counting Kit-8) was also performed on the same embodiment containing MSC-nFIB and MSC-nFIB-RAPA. In the embodiment of this example, nFIB-Rapamycin was used at a concentration of 5 μg/mL. The graph of FIG. 3B shows that in this embodiment neither the inclusion of nFIB or the nFIB-RAPA in the MSC affected cell viability and proliferation. The CCK-8 assay shown in FIG. 3B proves that in all nanomaterial-stem cells compositions, either unloaded (black bars) or loaded with the molecule drug Rapamycin (RAPA, silver bars), that the stem cells remain viable and continue to proliferate and they are metabolically active, as indicated by the increase of the optical density (ODs) from 24 to 48 hours. Here the stem cells are infrapatellar fat pad-derived mesenchymal stem cells (IFP-MSC). Furthermore, the embodiment maintains good stability and is viable for at least 120 hours in vitro in the culture solution (FIG. 3C).

### Example 3: MSC-nFIB and MSC-nFIB-RAPA Immunophenotype

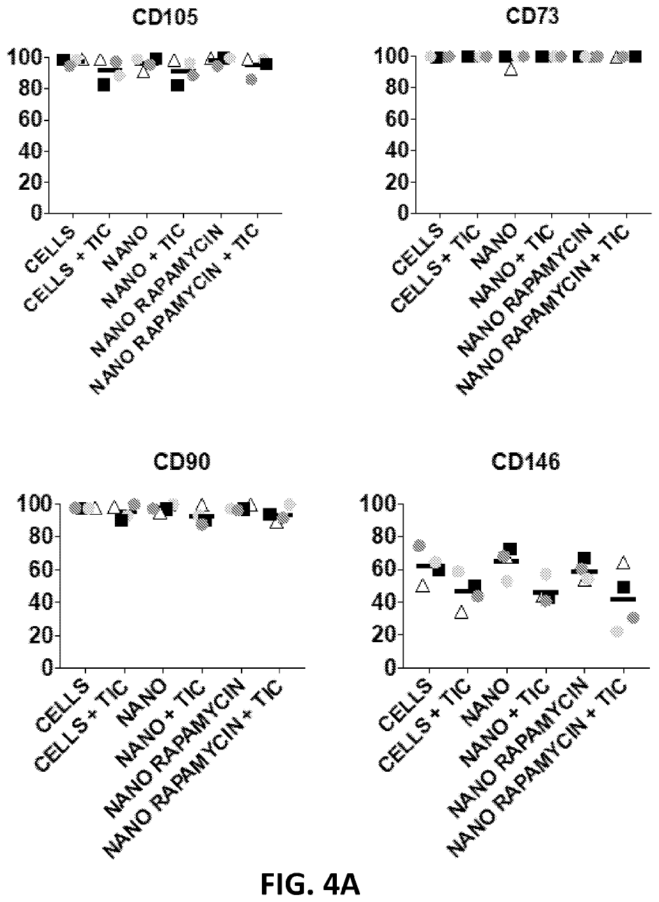

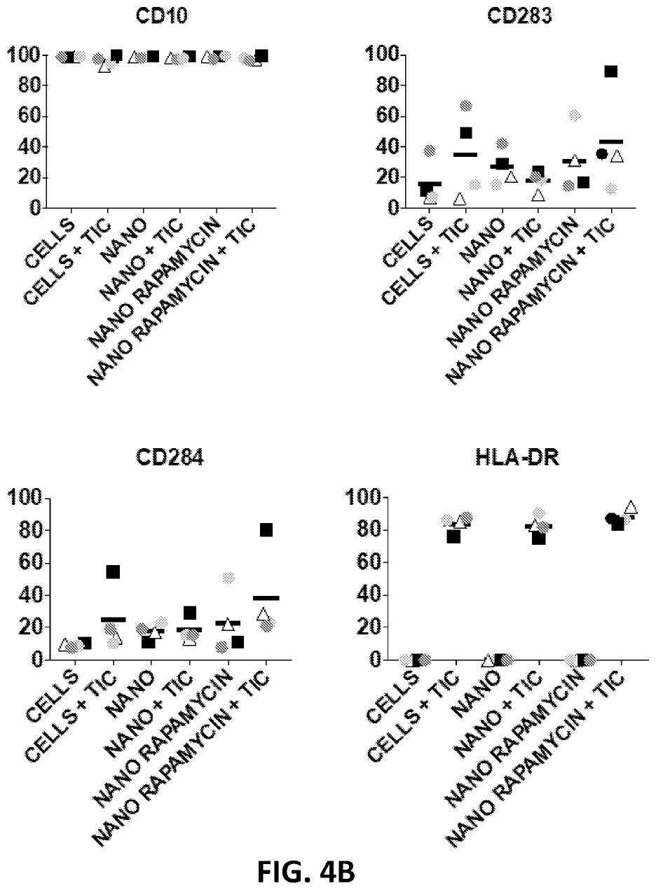

Naïve and TIC-induced IFP-MSC containing either nFIB or nFIB-RAPA, prepared as in the Example 2, were screened for their immunophenotype. TIC-induced cultures were primed with TIC inflammatory/fibrotic cocktail (15 ng/ml TNFα, 10 ng/ml IFNγ, 10 ng/ml CTGF) for 72 h. Flow cytometric analysis (FIGS. 4A and 4B) was performed on 2.0×10⁵ naïve and induced cells labelled with monoclonal antibodies specific for: CD73, CD90, CD105 (MSC markers), CD10, CD146 (immunomodulatory markers), HLA-DR, CD283, CD284. Results show that in this embodiment IFP-MSC are stable and the inclusion nFIB or nFIB-RAPA doesn't affect the expression of MSC markers, immunomodulatory markers and HLA-DR. Of note, CD283 and CD284 MSC polarization markers show slightly increased expression in the embodiment MSC-nFIB-RAPA previously induced with TIC cocktail.

### Example 4: Effects of MSC-nFIB-RAPA In Vitro-Inhibition of Human Cytotoxic T Cells

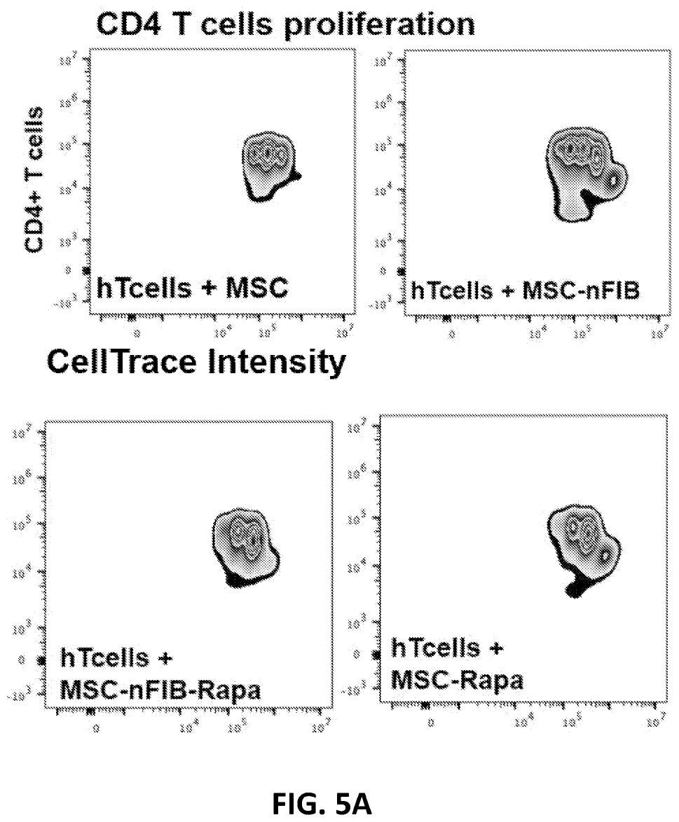

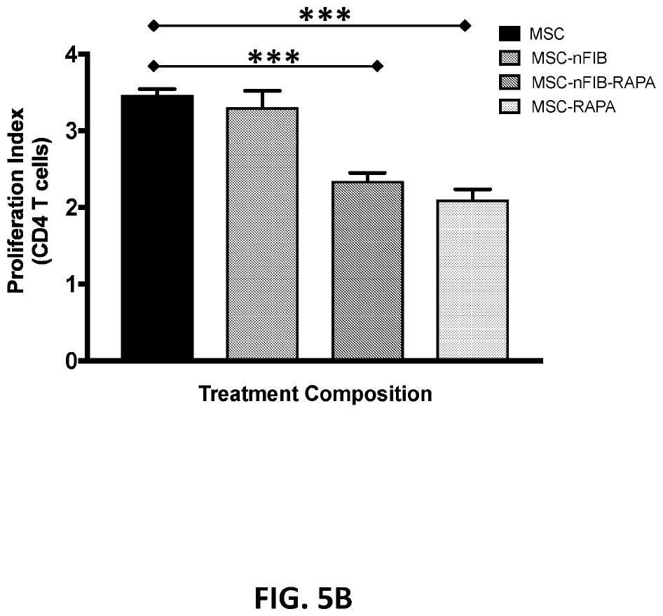

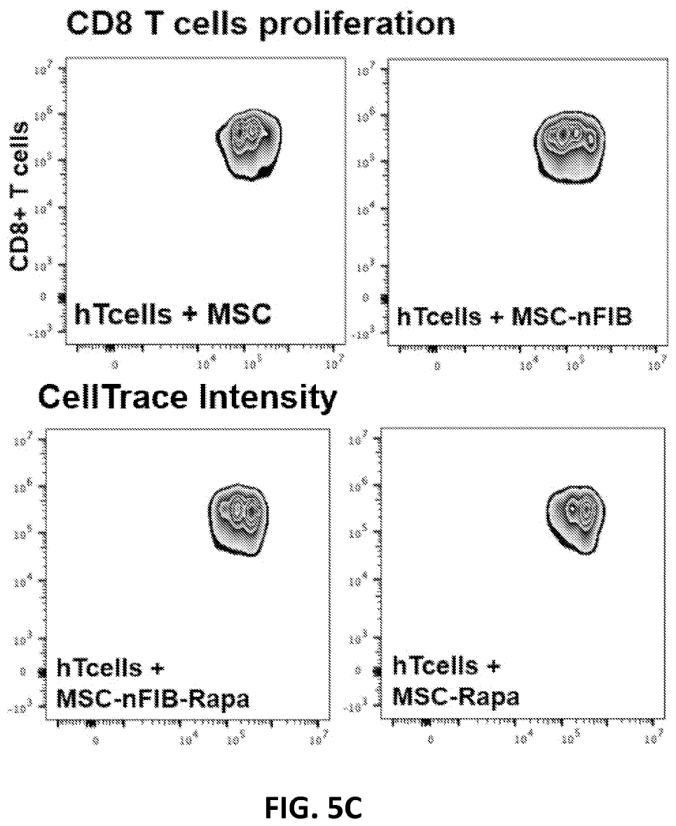

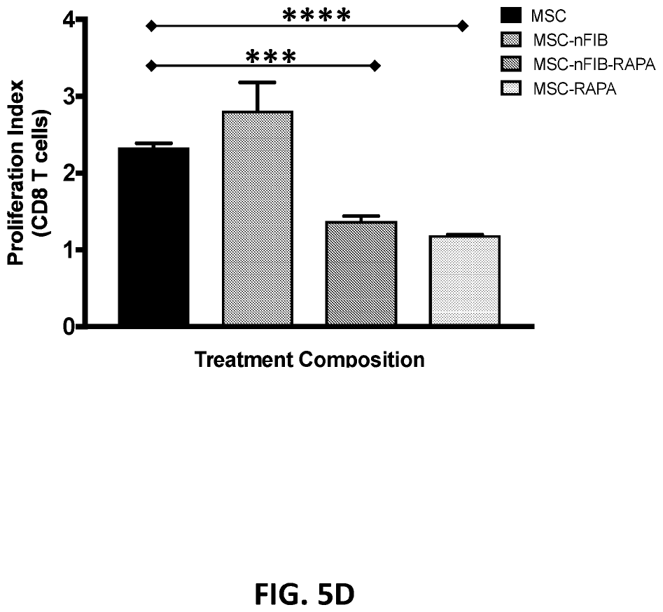

Human T cells were cultured in presence of MSC-nFIB-RAPA prepared as in Example 1, and they were activated with anti CD3/CD28 and IL2 mixture. In this example, the embodiment contains MSC-nFIB-RAPA obtained with a RAPA concentration of 1 μg/mL and the MSC were UC-MSC. Embodiments containing MSC or MSC-nFIB without RAPA were also cocultured with the T cells and used as controls. After 4 days of coculture, T cells were harvested and stained for flow cytometry analysis. The proliferation of CD4+ T cells (FIG. 5A) and CD8+ T cells (FIG. 5C) was assessed by CellTrace probe dilution. In FIG. 5A and FIG. 5C the stem cells are umbilical cord derived MSC. Stem cells alone (MSC) and unloaded nanomaterial-stem cell compositions (MSC-nFIB) are used as controls for Rapamycin-loaded nanomaterial-stem cell compositions (MSC-nFIB-RAPA). Graphs in FIGS. 5B and 5D report the proliferation index (calculated with FlowJo software) for live CD4+ T cells and live CD8+ T cells, respectively. The results of FIG. 5B and FIG. 5D refer to live CD4+ proliferated cells and show that drug-loaded nanomaterial-stem cells (MSC-nFIB-RAPA) can reduce CD4+ T cell proliferation in vitro, when compared to stem cells alone or stem cells with nanomaterial without drug loading. Results showed that the proliferation of both T cells populations was inhibited by the MSC-nFIB-RAPA composition, whose effect is stronger and more significant than the effect of the other compositions used in this example. In other examples, not shown, the concentration of the compositions would be different and adjustable for stronger or weaker T cell proliferation inhibition.

### Example 5: Effects of MSC-nFIB-RAPA In Vitro-Expansion of Regulatory T Cells

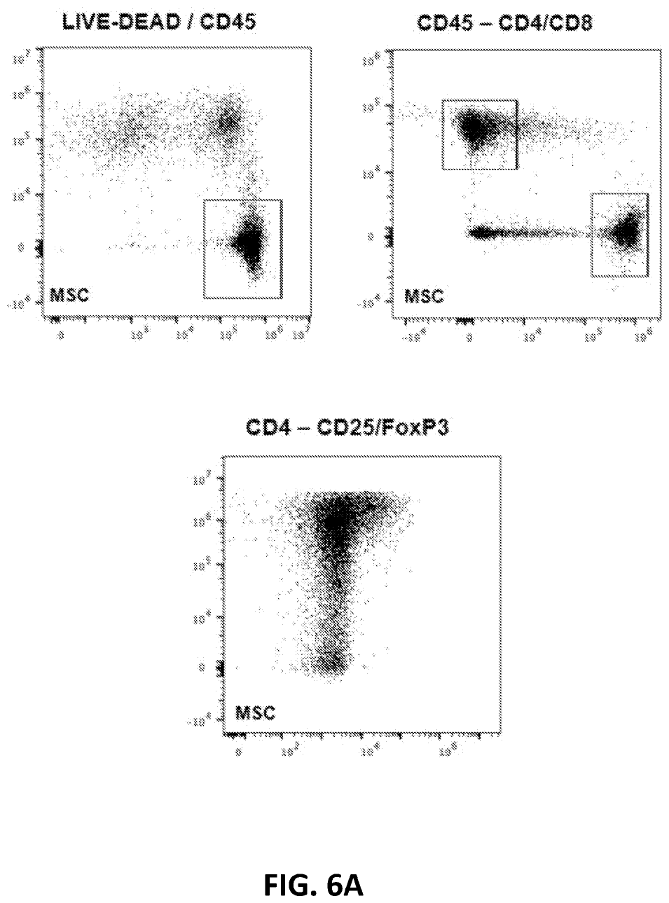

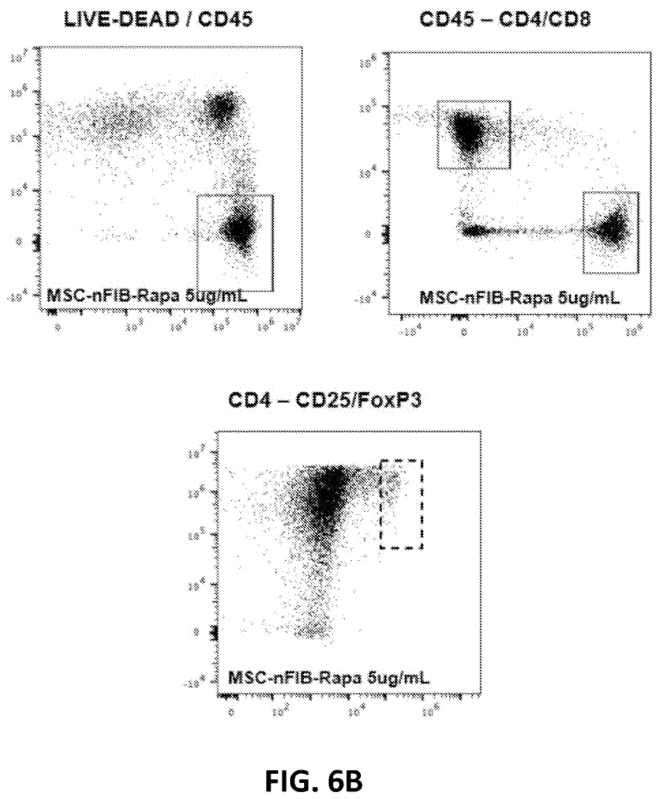

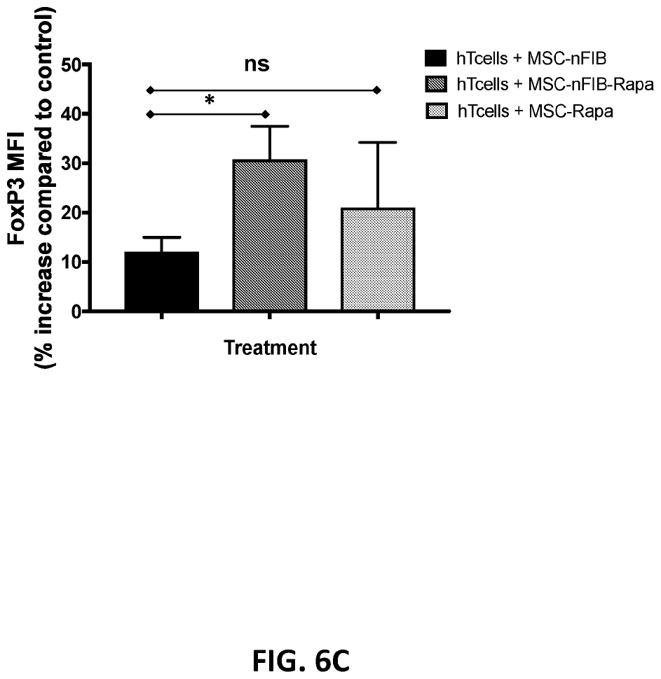

In an experiment similar to that reported in Example 4, human regulatory T cells (Treg) were also stained and analyzed by flow cytometer. Treg were identified by selecting live cells positive for markers against CD45, CD4, CD25 and FoxP3. T cells were activated via anti-CD3/CD28 and Interleukin 2 treatment. FIG. 6A and FIG. 6B show the gating strategy adopted for identifying Treg in those cultures treated with MSC (control) and with MSC-nFIB-RAPA, respectively. In FIG. 6B the dotted line marks a population of CD4+ CD25+ FoxP3+ Tregs observed expanded in the presence of MSC-nFIB-Rapa in comparison with FIG. 6A. The graph in FIG. 6C reports Treg expansion as percentage of mean fluorescent intensity (MFI) of FoxP3 positive cells respect to control, confirming the ability of MSC-nFIB-RAPA composition to induce higher Treg expansion than MSC-nFIB and MSC-RAPA compositions. The results in FIG. 6C show that the Rapamycin-loaded nanomaterial-stem cells composition (MSC-nFIB-RAPA) promotes Treg expansion even better than the clinically utilized therapeutic agent Rapamycin alone (prepared in methanol solution).

### Example 6: Preparation of Fluorescent MSC-nFIB Composition for In Vivo Biodistribution Study and Imaging after Intravenous Infusion

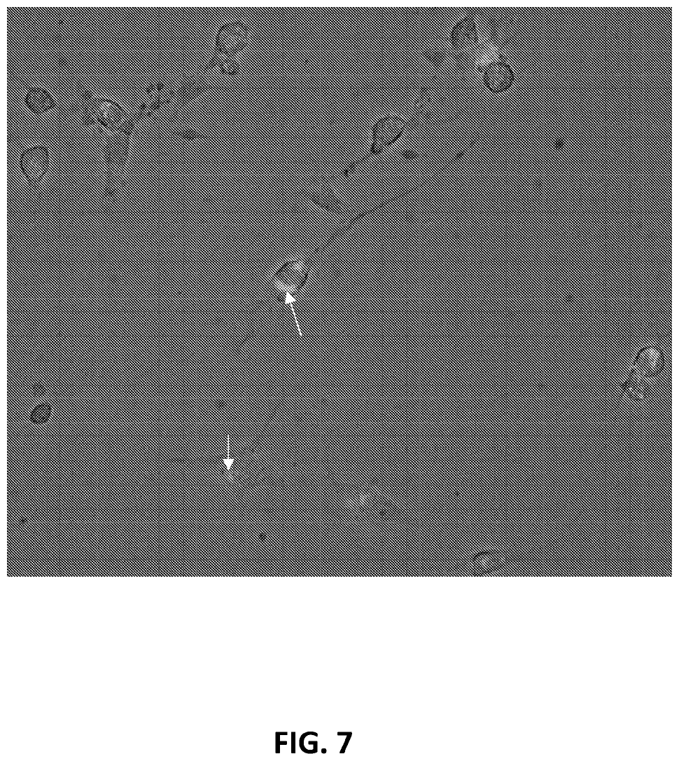

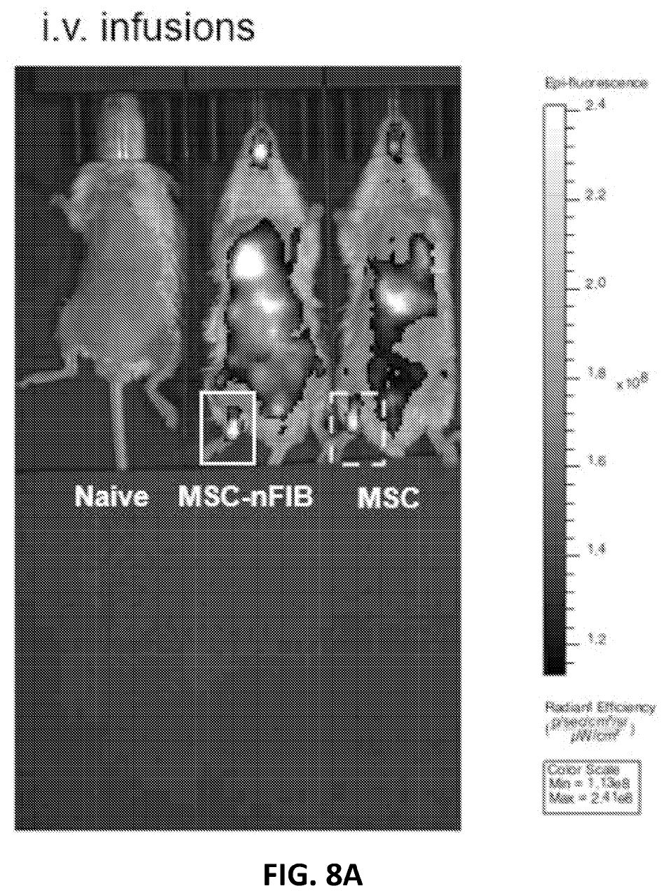

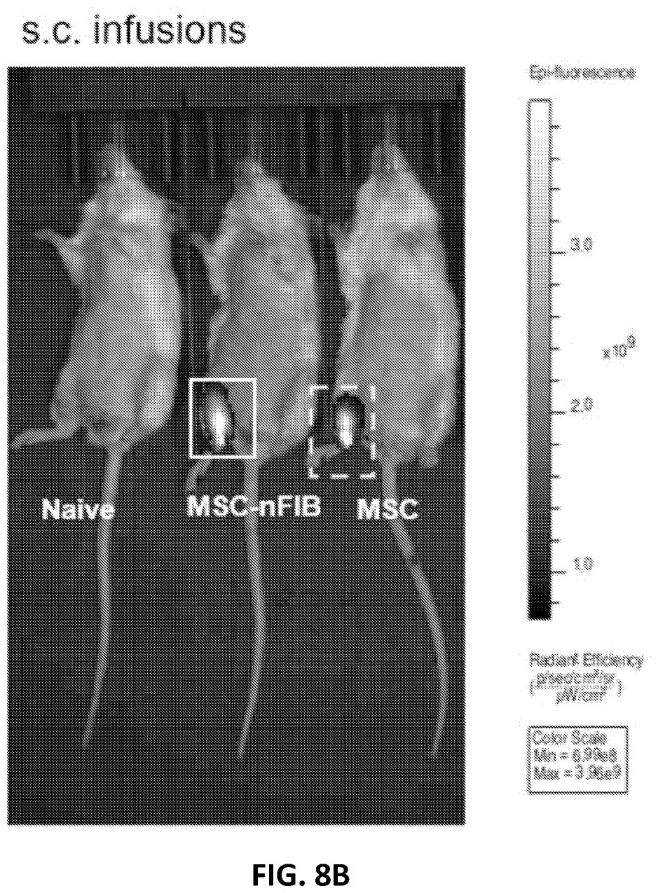

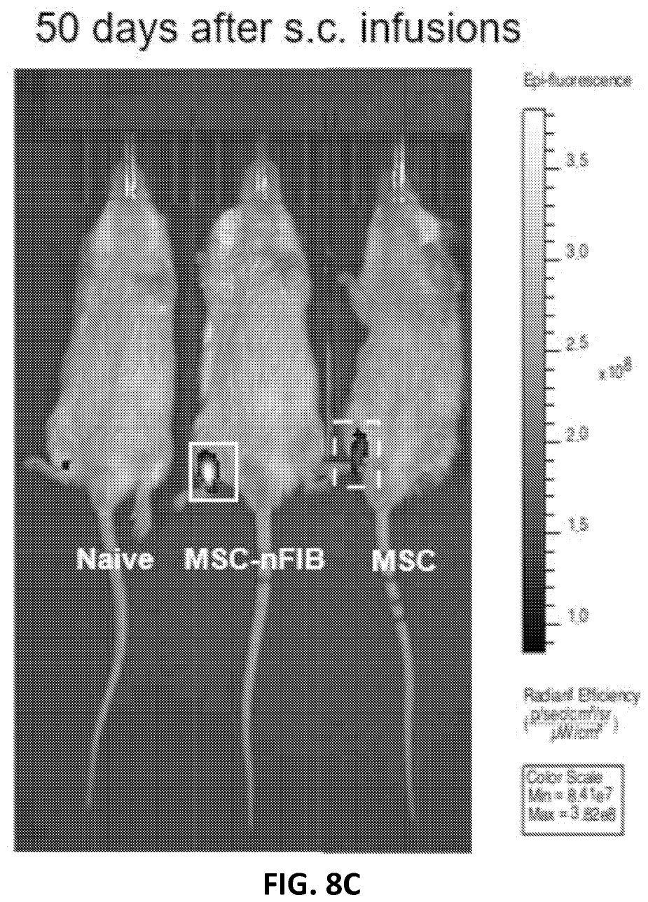

In this example, a preferred embodiment containing fluorescently labeled MSC-nFIB was prepared. Briefly, nFIB were labeled with a near-infrared DiR core-labeling dye as described in Example 1 and added to a solution of 65% confluent adherent UC-MSC. The MSC and the nFIB-DiR were incubated overnight. After several washes the MSC were harvested and stained with the far-red DiD cellular probe (5 to 10 μM/10⁶ cells) to obtain MSC (DiD)-nFIB (DiR) composition. The fluorescent composition was seeded again on cell culture plate and soon observed by fluorescent microscopy. Image in FIG. 7 proved that the nFIB were stably combined with MSC and retained inside the cells. For FIG. 7, aliquots of this composition that were not implanted in vivo were seeded in in vitro culture and observed in adhesion 24 hours later. Nanomaterials of FIG. 7 are nFIB with a fluorescent core-labeling dye and stem cells are UC-MSC stained with a nucleus probe (Hoechst staining). Therefore, fluorescently labeled MSC-nFIB composition was used to study the biodistribution and stability of the preferred embodiments in vivo. Balb/C mice were treated with Lipopolysaccharide (LPS) injection in the right foot paw to induce localized inflammation. MSC-nFIB compositions were subsequently administered via intravenous infusion and 24 hours later, imaging was performed via In Vivo Imaging System (IVIS) to detect the distribution of the MSC-nFIB composition. This imaging (FIG. 8A) showed accumulation and localization of the MSC-nFIB composition (white rectangle) and MSC alone (white dotted rectangle) at the site of inflammation.

### Example 7: Preparation of Fluorescent MSC-nFIB Composition for In Vivo Biodistribution Study and Imaging after Subcutaneous Infusion

As in Example 6 (FIG. 8A), in this experiment inflammation was induced in the right foot paw of Balb/C mice. Fluorescently labeled MSC-nFIB composition were subsequently administered via subcutaneous injection. Imaging via IVIS (FIG. 8B) showed accumulation and localization of the MSC-nFIB composition (white square) and MSC alone (white dotted square) at the site of inflammation. In vivo imaging of the same mice performed 50 days after the subcutaneous administration of the MSC-nFIB composition and MSC alone (FIG. 8C), demonstrated the long-term persistence of accumulated and localized MSC-nFIB composition (white square) and MSC alone (white dotted square) at the site of inflammation.

### Example 8: Aggregation of MSC-nFIB with Pancreatic Islets

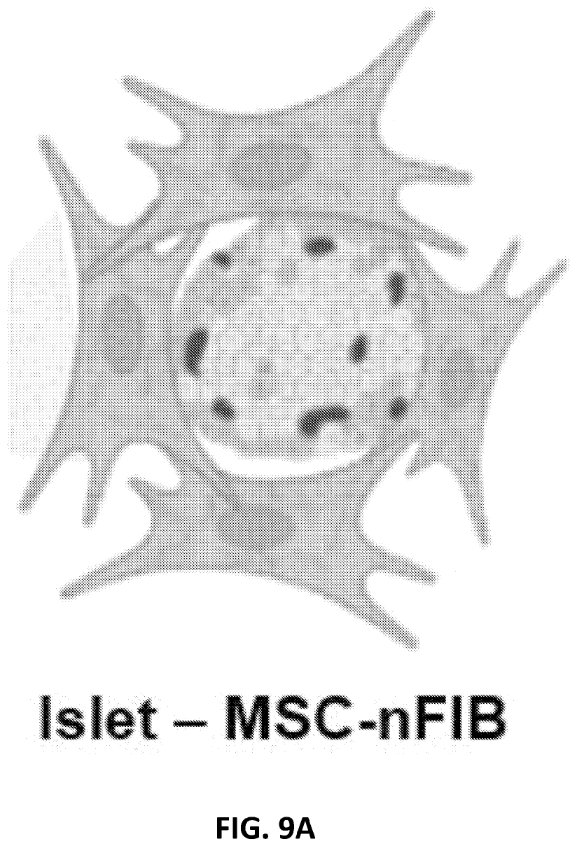

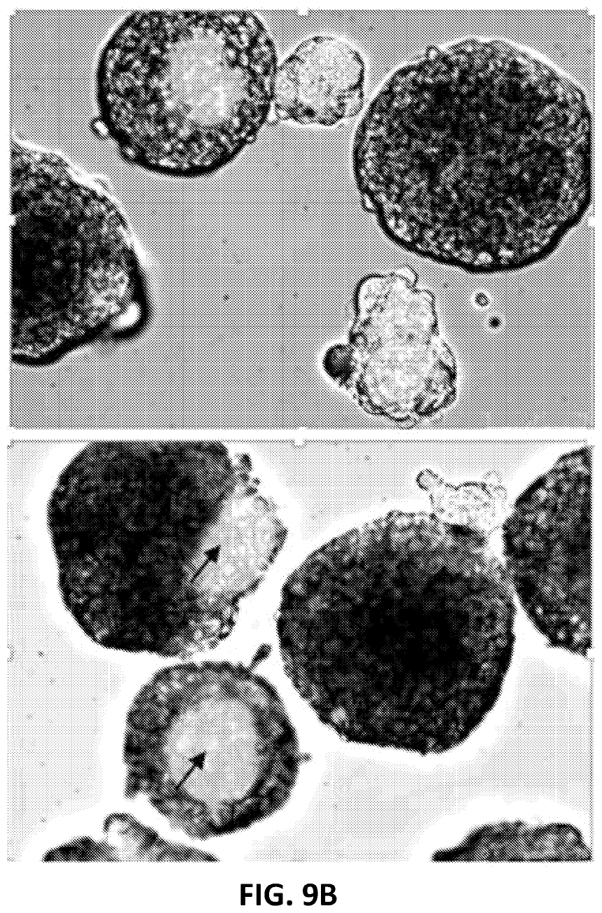

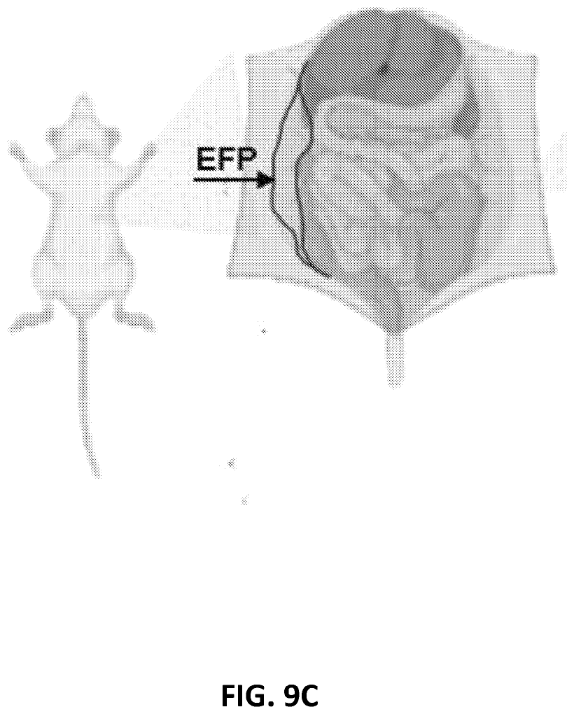

FIG. 9A is a schematic of the MSC-nFIB composition and its use as drug delivery system in pancreatic islet transplantation: the MSC-nFIB composition is pre-aggregated with isolated pancreatic islets to facilitate contact and localization at the site of implant (in this example, the site of implant is the epidydimal fat pad). Freshly isolated pancreatic islets from healthy C57/BL6 mice, were treated with fluorescently labeled MSC-nFIB composition using an islet cells/MSC ratio of 5. The mixture was incubated in the islet solution overnight in AggreWell™ plates to force the formation of aggregates like schematized in FIG. 9A. Fluorescent microscopy image (FIG. 9B) showed MSC nicely aggregated on the surface of the islets (brighter spots in FIG. 9B). As reported in the next example, these compositions, islet-MSC-nFIB or islet-MSC, were harvested and implanted in the epididymal fat pad (EFP) of diabetic C57/BL6 mice (schematic in FIG. 9C).

### Example 9: Use of MSC-nFIB Compositions in Pancreatic Islet Transplantation

In this example, C57/BL6 mice were rendered diabetic via injection of streptozotocin. Diabetic mice were implanted in the EFP with 750 islet equivalents isolated from healthy C57/BL6 mice and pre-aggregated with fluorescently labelled MSC or MSC-nFIB, as described in Example 8. Mice receiving untreated islets and islets+nFIB were also used as controls. In vivo imaging (IVIS) of transplanted mice were performed every day to detect both DiD (identifying the MSC) and DiR (identifying the nFIB) channels in the EFO site.

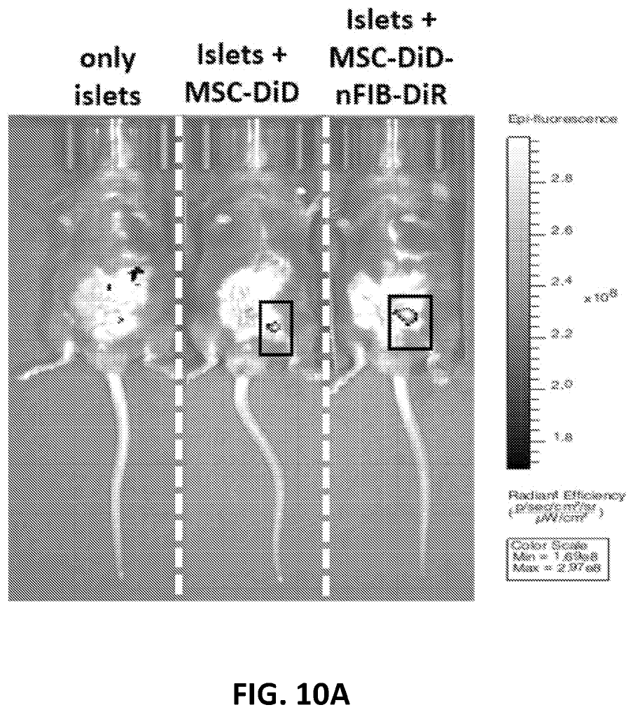

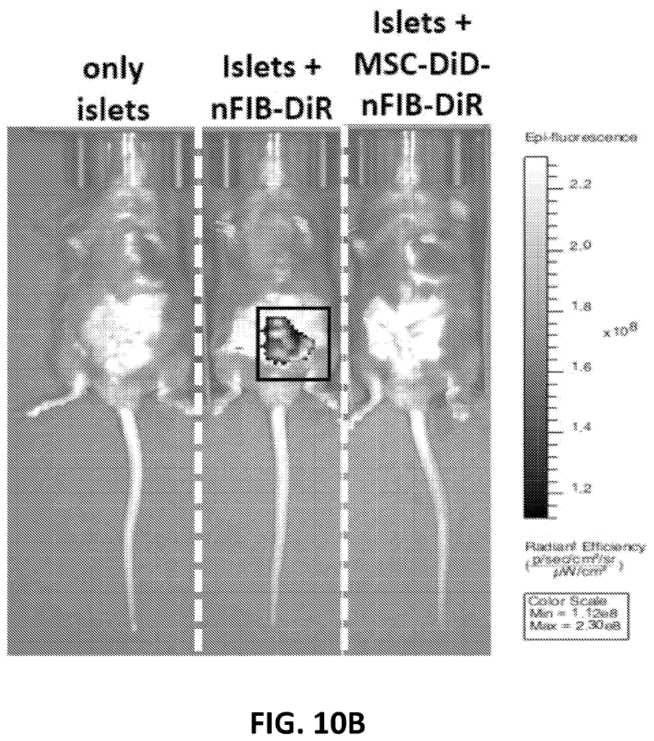

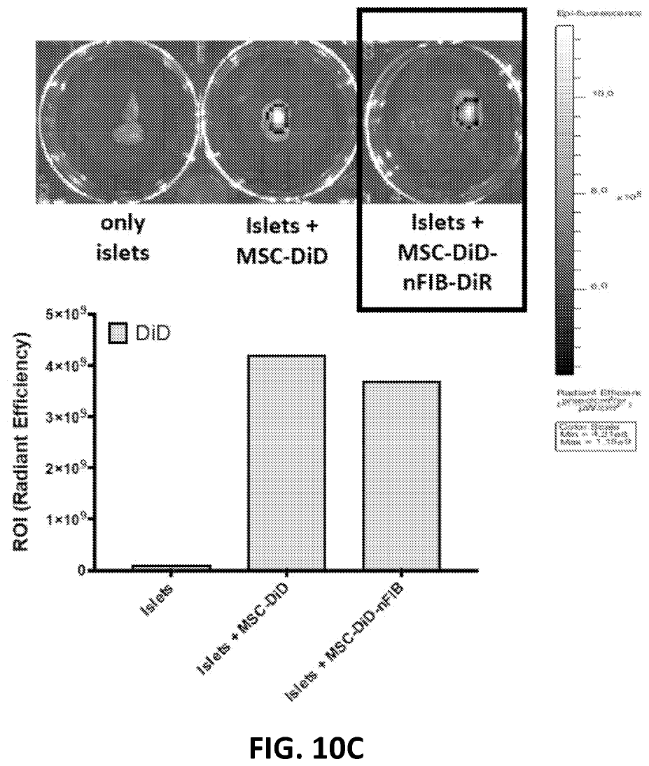

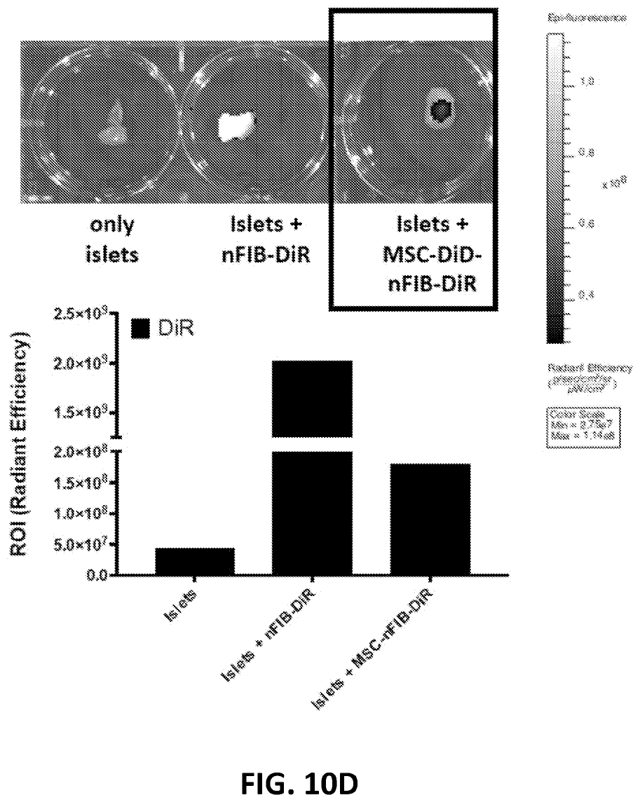

FIG. 10A shows in vivo imaging for DiD channel of implanted mice at post operative day 7. The MSC or MSC-nFIB were stained with a fluorescent probe (DiD) before aggregation with islets. Images demonstrated localization of the MSC and MSC-nFIB composition in the EFP. The outcomes of this experiment were also confirmed by ex vivo imaging of the EFP resected from the implanted mice (FIG. 10C) and by evaluating the DiD intensity of the region of interest (ROI) as shown in the graph of FIG. 10C.

On the other hand, in FIG. 10B the in vivo imaging for DiR channel of implanted mice is reported for the same set of mice of FIG. 10A. The images showed localization of the nFIB (DiR) in the EFP at post operative day 7, but DiR positivity in the MSC-nFIB composition could not be detected with in vivo imaging (due to signal from tissue too deep in the body), but it was detected by ex vivo imaging in the resected tissue (FIG. 10D). The ROI measurements confirmed detection of DiR signal in the EFP implanted with islets and nFIB, but also in the EFP implanted with pre-aggregated islet-MSC-nFIB.

Overall, in this example, MSC and MSC-nFIB compositions were demonstrated to accumulate and be retained for days at the site of transplant.

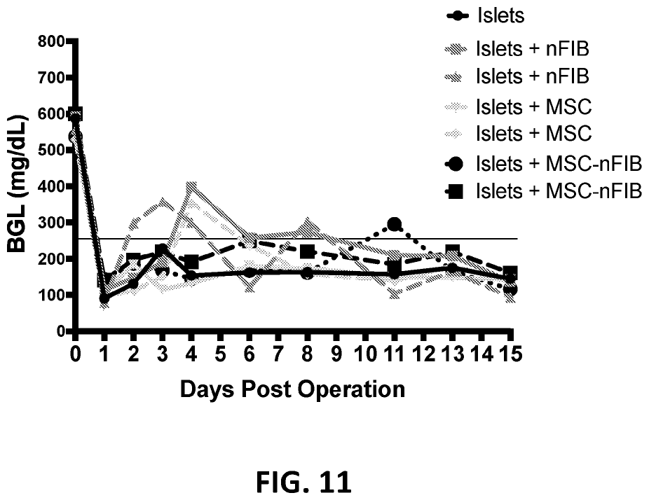

Moreover, the blood glucose measurements (reported in FIG. 11) for the mice transplanted with islets alone or co-transplanted with nFIB and with islets pre-aggregated with MSC and MSC-nFIB, showed that all the animals reversed diabetes (blood glucose level <250 mg/dL). This indicates that MSC-nFIB compositions do not negatively affect the insulin production and the beta cell function of islet grafts for the duration of follow-up.

Although the technology herein has been described with reference to embodiments, it is to be understood that these embodiments are merely illustrative of the principles and applications of the present technology. It is therefore to be understood that numerous modifications may be made to the illustrative embodiments and that other arrangements may be devised without departing from the spirit and scope of the present technology as defined by the appended claims.

### REFERENCES

Anthony D. F., Shiels P. G. (2013), Exploiting paracrine mechanisms of tissue regeneration to repair damaged organs, Transplant. Res. 2, 10.
Berman D. M. et al. (2010), Mesenchymal Stem Cells Enhance Allogeneic Islet Engraftment in Nonhuman Primates Diabetes 59, 2558-2568.
Brubaker, C. E., Velluto, D., Demurtas, D., Phelps, E. A., Hubbell, J. A. (2015), Crystalline oligo(ethylene sulfide) domains define highly stable supramolecular block copolymer assemblies. ACS Nano 9, 6872-6881.
Christian D. A., Cai S., Garbuzenko O. B., Harada T., Zajac A. L., Minko T., Discher D. E. (2009), Flexible filaments for in vivo imaging and delivery: persistent circulation of filomicelles opens the dosage window for sustained tumor shrinkage, Mol. Pharm. 6 (5) 1343-1352.
De Miguel M. P., Fuentes-Julian S., Blazquez-Martinez A. et al (2012), Immunosuppressive properties of mesenchymal stem cells: advances and applications. Curr Mol Med 12:574-591.
Ding Y., Xu D., Feng G., Bushell A., Muschel R. J., Wood K. J. (2009) Mesenchymal stem cells prevent the rejection of fully allogenic islet grafts by the immunosuppressive activity of matrix metal-loproteinase-2 and -9. Diabetes 58:1797-1806.
Duncan R., Gaspar R. (2011) Nanomedicine(s) under the microscope. Mol Pharm 8 (6): 2101-2141.
Eggenhofer E., Renner P., Soeder Y. et al. (2011), Features of synergism between mesenchymal stem cells and immunosuppressive drugs in a murine heart transplantation model. Transpl Immunol 25:141-14732.
Fox J. M., Chamberlain G., Ashton B. A., Middleton J. (2007), Recent advances into the understanding of mesenchymal stem cell trafficking. Br J Haematol 137:491-502.
Gao Z., Zhang L., Hu J., Sun Y., (2013), Mesenchymal stem cells: a potential targeted-delivery vehicle for anti-cancer drug loaded nanoparticles. Nanomedicine: NBM 2013; 9:174-184.
Girdlestone J. et al. (2015), Enhancement of the immunoregulatory potency of mesenchymal stromal cells by treatment with immunosuppressive drugs Cytotherapy 17, 1188-1199.
Lanzoni G., Linetsky E., Correa D., Cayetano S. R., Alvarez R. A., Kouroupis D., et al. (2021), Umbilical cord mesenchymal stem cells for COVID-19 acute respiratory distress syndrome: A double-blind, phase 1/2a, randomized controlled trial. STEM CELLS Transl Med. 10, 660-673.
Le Blanc K., Samuelsson H., Lonnies L., Sundin M., Ringden O. (2007), Generation of immunosuppressive mesenchymal stem cells in allogeneic human serum. Transplantation 84:1055-1059.
Sadhukha T., O'Brien T. D., Prabha S. (2014), Nano-engineered mesenchymal stem cells as targeted therapeutic carriers. J of Controlled Release 196:243-251.
Uccelli A., Moretta L., Pistoia V. (2008), Mesenchymal stem cells in health and disease. Nat Rev Immunol 8:726-736.
Velluto D., Bojadzic D., De Toni T., Buchwald P., Tomei A. A. (2021), Drug-integrating amphiphilic nanomaterial assemblies: 1. Spatiotemporal control of cyclosporine delivery and activity using nanomicelles and nanofibrils. J Control Release 329, 955-970.
Velluto D., Demurtas D., Hubbell J. A. (2008), PEG-b-PPS diblock copolymer aggregates for hydrophobic drug solubilization and release: cyclosporin A as an example. Mol Pharm 5, 632-642.
Velluto D., Ricordi C. (2017), Nanotechnology Advances in Drug Delivery. NanoWorld J 3 (S2): S9-S17.
Yagi H., Soto-Gutierrez A., Parekkadan B., Kitagawa Y, Tompkins R. G., Kobayashi N., Yarmush M. L. (2010), Mesenchymal stem cells: mechanisms of immunomodulation and homing. Cell Transplant. 19, 667-679.

What is claimed is:

1. A nanomaterial-stem cell composition comprising:
a poly(ethylene glycol) oligo(ethylene sulfide) (PEG-OES) based fibril nanomaterial (nFIB) incorporated into one or more stem cells.

2. The composition of claim 1, wherein the one or more stem cells are mesenchymal stem cells (MSC).

3. The composition of claim 2, wherein the MSC are derived from an umbilical cord or infrapatellar fat pad.

4. The composition of claim 1, wherein the nFIB is comprised of a poly(ethylene glycol) (PEG) block molecular weight of 500-4,600.

5. The composition of claim 1, wherein the nFIB is comprised of an oligo(ethylene sulfide) (OES) with a degree of polymerization from 2 to 20.

6. The composition of claim 1, wherein the nFIB is comprised of a PEG block molecular weight of about 2000 and an OES block degree of polymerization of about 5.

7. The composition of claim 1, wherein the nFIB is PEG₄₄-OES₅.

8. The composition of claim 1, further comprising a molecule of interest.

9. The composition of claim 8, wherein a mass ratio of the nFIB to the molecule of interest is 10-30.

10. The composition of claim 9, wherein a mass ratio of the nFIB to the molecule of interest is 20.

11. The composition of claim 8, wherein solubility of the molecule of interest into the nFIB is about 0.1 mg/ml to about 20 mg/ml.

12. The composition of claim 11, wherein solubility of the molecule of interest into the nFIB is about 3 mg/ml.

13. The composition of claim 8, wherein the molecule of interest is a drug or probe.

14. The composition of claim 13, wherein the probe is an imaging probe.

15. The composition of claim 8, wherein the molecule of interest is present in the mesenchymal stem cells.

16. The composition of claim 8, wherein the nFIB comprises a non-covalently attached molecule of interest.

17. The composition of claim 8, wherein the molecule of interest is covalently attached to the nFIB.

18. The composition of claim 8, wherein the molecule of interest is an anti-cancer drug.

19. The composition of claim 8, wherein the molecule of interest is radioactive.

20. The composition of claim 1, further comprising pancreatic islets or stem cell-derived islets.

21. The composition of claim 20, wherein the pancreatic islets are aggregated with the MSC-nFIB.

22. The composition of claim 1, wherein the nFIB is about 5 nm in diameter.

23. The composition of claim 1, wherein the nFIB is about 500 nm to 1.5 μm in length.

24. The composition of claim 1, wherein the nFIB is about 1.0 μm in length.

25. The composition of claim 1, wherein the nFIB minimally or does not alter the MSC phenotype or viability.

26. The composition of claim 8, wherein the molecule of interest is a hydrophobic therapeutic molecule.

27. The composition of claim 26, wherein the hydrophobic therapeutic molecule is rapamycin (RAPA).

28. The composition of claim 27, wherein the concentration of rapamycin between about 1.0 to 10.0 g/mL.

29. The composition of claim 28, wherein the nFIB and rapamycin minimally or does not alter the MSC phenotype or viability.

30. The composition of claim 1, wherein the composition is utilized as a therapeutic, diagnostic, drug delivery mechanism, or extended release drug delivery mechanism.

31. The composition of claim 30, wherein the therapeutic is rapamycin (RAPA).

32. The composition of claim 30, wherein the diagnostic is a probe.

33. A method for preparing MSC-nFIB-molecule of interest comprising
providing a PEG-OES copolymer;
suspending the PEG-OES copolymer and a molecule of interest in water or an organic solvent;
removing unloaded molecule of interest;
adding the PEG-OES-molecule of interest to MSC; and
incubating the PEG-OES-molecule of interest and the MSC together.

34. The method of claim 33, wherein the molecule of interest is rapamycin (RAPA).

35. The method of claim 33, wherein the molecule of interest is a probe.

36. The method of claim 36, wherein the probe is fluorescent.

37. The method of claim 33, wherein the MSC are derived from an umbilical cord or infrapatellar fat pad.

38. The method of claim 33, wherein the incubating occurs for 24 hours.

39. A method of treating a condition in a subject comprising administering MSC-nFIB-molecule of interest to the subject in need thereof.

40. The method of claim 39, further comprising pancreatic islet cells.

41. The method of claim 40, wherein the condition is diabetes.

42. The method of claim 39, wherein the molecule of interest is rapamycin (RAPA) and MSC-nFIB-RAPA is formed.

43. The method of claim 42, wherein the MSC-nFIB-RAPA reduces the proliferation of cytotoxic T cells when the cytotoxic T cells are in proximity to the MSC-nFIB-RAPA or derivatives.

44. The method of claim 42, wherein the MSC-nFIB-RAPA expands regulatory T cells when the regulatory T cells are in proximity to the MSC-nFIB-RAPA or derivatives.

45. The method of claim 39, wherein the MSC-nFIB-molecule of interest is injected into the subject as a therapeutic concentration.

46. The method of claim 39, wherein the MSC-nFIB-molecule of interest reaches a site of inflammation after intravenous infusion.

47. The method of claim 39, wherein the MSC-nFIB-molecule of interest are localized at a site of inflammation or implantation.

48. The method of claim 39, wherein the MSC-nFIB-molecule of interest release nFIB and/or a drug over time.

49. The method of claim 39, wherein the MSC-nFIB-molecule of interest are aggregated with pancreatic islet cells or other cells.

50. The method of claim 39, wherein the MSC-nFIB-molecule of interest are co-transplanted in a confined space and remain in or in proximity to such confined space for at least 7 days.

51. The method of claim 39, wherein the MSC-nFIB-molecule of interest are aggregated on a surface of pancreatic islets and the functionality of the islets in vivo is not affected.

52. The method of claim 39, wherein the MSC-nFIB-molecule of interest are intended for therapeutic use.

53. The method of claim 39, wherein the MSC-nFIB-molecule of interest, further provide a diagnostic ability.

54. The method of claim 39, wherein the MSC-nFIB-molecule of interest provide drug delivery.

55. The method of claim 39, wherein the MSC-nFIB-molecule of interest provide extended release of a drug.

56. The method of claim 39, wherein the MSC-nFIB-molecule of interest modulate immune functions.

57. The method of claim 39, wherein the MSC-nFIB-molecule of interest improve the outcomes of a transplant.

58. A kit comprising:
instructions for using a nanomaterial-stem cell composition comprising an nFIB incorporated into MSC; and
the nanomaterial-stem cell composition comprising an nFIB incorporated into MSC.

59. A kit comprising:
instructions for performing the method of any one of claims 32-42; and
a nanomaterial-stem cell composition comprising an nFIB incorporated into MSC.
